(19) United States
(12) Patent Application Publication
(10) Pub. No.: US 2016/0346603 A1
(43) Pub. Date: Dec. 1, 2016

(54) Pulmonary System Resistance Training Apparatus and Methods

(71) Applicant: PULMONARI, LLC, Wilmington, DE (US)

(72) Inventors: Christopher I. HALLIDAY, Phoenixville, PA (US); Jeffrey DIFRANCESCO, Riverton, NJ (US)

(73) Assignee: PULMONARI, LLC, Wilmington, DE (US)

(21) Appl. No.: 15/116,744

(22) PCT Filed: Feb. 10, 2015
(86) PCT No.: PCT/US15/15167

§ 371 Date: Aug. 4, 2016

Related U.S. Application Data
(60) Provisional application No. 61/937,716, filed on Feb. 10, 2014.
(60) Provisional application No. 62/047,859, filed on Sep. 9, 2014.
(60) Provisional application No. 62/075,998, filed on Nov. 6, 2014.

(51) Int. Cl. A63B 23/18; A61M 16/20; A63B 21/00

(52) U.S. Cl. CPC A63B 23/18; A63B 21/00189; A61M 16/208; A63B 2220/12; A63B 2220/51; A63B 2230/06; A63B 2230/205; A63B 2230/42; A61M 2205/3303; A61M 2205/0294; A61M 2205/52; A61M 2205/8206; A61M 2230/06; A61M 2230/42; A61M 2230/432; A61M 2230/46; A61M 2230/63

(57) ABSTRACT

A respiratory exerciser having a hollow body, where the hollow portion is in communication with a porous or non-porous material and with an opening that provides access for a user to breathe through the device.

### CROSS-REFERENCE TO RELATED APPLICATIONS

This application claims the benefit of U.S. Provisional Patent Application Nos. 61/937,716, filed 10 Feb. 2014 entitled “Pulmonary System Resistance Training Apparatus and Methods”, 62/047,859, filed 9 Sep. 2014 entitled “Pulmonary System Resistance Training Apparatus and Methods”, and 62/075,998 filed 6 Nov. 2014 entitled “Pulmonary System Resistance Training Apparatus and Methods of Interacting With Technological Devices”, all of which is incorporated by reference herein in its entirety.

### BACKGROUND OF THE INVENTION

Respiratory exercisers, in general, are designed for purposes including the assistance of patients with lung problems due to chronic obstructive pulmonary disease (COPD) or postoperative loss of spontaneous deep breaths. The types of devices vary-one type may provide inhalation resistance while a second type may provide exhalation resistance. The devices are designed so that resistance can be varied to adjust for the changing strength or weakness of the user.

Other respiratory exercise devices include large and/or complex multi-component assemblies of springs, valves, diaphragms notches and/or apertures, such as the devices shown in U.S. Pat. Nos. 6,726,598, 5,899,832 and 4,739,987, the contents of which are incorporated by reference in their entirety.

Other devices show exhalation through a porous component, as shown in U.S. Pat. No. 7,921,964 and WO 2009/106062, the entire contents of which are hereby incorporated by reference in their entirety. However, in these other devices, exhalation effort and pressure is sought to be minimized, and inhalation through the porous plastic is not possible due to valve restrictions to avoid drowning.

### SUMMARY OF THE INVENTION

A pulmonary exercise device including a T or L shaped frame having a center portion defining an interior space and a mouthpiece communicating with the interior space, a first attachment interface in fluid communication with the interior space, a second attachment interface connected to the center portion by an arm portion, a valve, a resistance member or measurement member in fluid communication with the interior space (e.g., a one way and/or adjustable one way valve) and supported by and removably coupled to the first and/or second attachment interfaces.

In certain embodiments, two or more of a valve, a resistance member or measurement member can be connected to, or placed in fluid communication with, the center portion. In certain such embodiments, one or both of the valve and resistance member or measuring member are supported by and non-removably connected to the frame at one or more contact points (e.g., distal and proximal ends of the valve member or resistance member). In certain such embodiments, one or both of the valve and resistance member or measuring member are elongated and supported by and removably connected to the frame at one or more contact points (e.g., distal and proximal ends of the valve member or resistance member).

In certain embodiments, the resistance member can be a porous plastic module, an aperture providing member, an adjustable valve (e.g., an adjustable one way valve), or a dead space providing member. In certain embodiments, the measurement member can be a mechanical or piezo electric measurement member configured to measure one or more of exhalation count, inhalation count, breath count, duration and/or force thereof. It is understood that the term “a breath” can be considered to encompass inhalation alone, exhalation alone, or a pair of an inhalation and exhalation together.

In one embodiment, the present invention includes a device body adapted to removably receive: (a) a one-direction valve having a cracking pressure of less than 10 psi; (b) a mouthpiece; and, (c) at least one other component selected from a one direction valve having a cracking pressure less than 10 psi, cassette, an adjustable valve and a dead space introducing member. In some embodiments, one or more of a valve, cassette or dead space introducing member further includes one or more of a pressure measurement member (e.g., a spirometer or piezoelectric circuit), GPS component, wireless communication circuitry, battery and programming to provide information concerning one or more of time, position, pressure and breaths to a distal receiver. Such a device may also include a memory for storage so that the information can be provided to a receiver (e.g., a smartphone or computer) at a later date.

In another embodiment, there is provided a method of measuring pulmonary fitness progression including the steps of wirelessly receiving a signal from a pulmonary exerciser, where the signal includes information concerning one or more of time of day, elapsed time, GPS location, breath count, breath duration, and breath force, storing the information in a memory, analyzing the information to determine one or more of breath count, average number of breaths, average number of breaths over a given time or distance, breath duration, average breath duration, average breath duration over a given time or distance, breath force, average breath force, and average breath force over a given time or distance and/or displaying that information to a user.

Additionally, the information can be analyzed and/or characterized as a function of one or more fitness indicia that does not directly relate to breathing (e.g., distance, time of day, elapsed time, heart rate, calories burned, instant pace, average pace over a distance, lap pace over a distance, location/position, elevation change, and elevation); and, displaying the information.

Additional methods of use and other embodiments are also disclosed.

### BRIEF DESCRIPTION OF THE DRAWINGS

Further details are explained below with the help of the examples illustrated in the accompanying drawings in which:

FIG. 1 shows a top view of a device;

FIG. 2 shows a bottom view of a device;

FIG. 3 shows a front view of a device;

FIG. 4 shows a mouthpiece view of a device;

FIG. 5 shows a right side view of a device;

FIG. 6 shows a left side view of a device; and

FIG. 7 shows a partially transparent view of a device including electronic module.

### DETAILED DESCRIPTION OF THE INVENTION

The present invention relates to respiratory exercise devices and, more particularly, to breathing exercise devices which promote proper or stronger inhalation and exhalation by the user. The disclosure of PCT U.S. Ser. No. 13/54494 (Published as WO 2014/028370) is hereby incorporated by reference in its entirety. As used herein, the general term “device of the present invention” or “present invention” is understood to include each of a frame, a cassette, valve, or dead space introducing member each alone or in any combination, as dictated by context and description.

As with other resistance training, inhalation and exhalation resistance training can increase athletic performance In certain instances, performance can be increased between 1 and 10 percent, most typically between about 2 and about 7 percent with about 5 percent not being an uncommon increase in physiologic/athletic performance. In certain embodiments, the present invention can be configured to provide one or more of flow resistance loading, pressure threshold loading or isocapnic hyperpnea. In certain embodiments, the present invention can be configured to provide one of flow resistance loading, pressure threshold loading or isocapnic hyperpnea and optionally provide an additional mode of operation (e.g., flow resistance and optionally pressure threshold and/or isocapnic hyperpnea). In one embodiment, the present invention can be configured to provide two or more of: flow resistance loading, pressure threshold loading or isocapnic hyperpnea.

In certain embodiments, the present invention includes a generally linear device body, or in certain embodiments a W-shaped, U-shaped, T-shaped or L-shaped body or frame, in connection with a mouthpiece. None, all, or a portion of the overall device body may be composed of a porous material, e.g., porous ceramic, porous metal or porous plastic material. In certain embodiments, the weight of the present invention is sufficiently low so as to permit a user of the present invention to hold the present invention in the user's mouth solely by use of a mouthpiece. In such embodiments, the weight of the present invention can minimize jaw fatigue while the user is biting down on the mouthpiece and using the present invention, and also permits hands free use, e.g., during running, cycling, stair climbs, or other exercises involving cardiovascular or muscle training

Porous portions of the present invention (e.g., all or portions of the device body and/or cassette, and/or valve and/or a dead space introducing member) include pores or interconnected cavities permitting gaseous or fluidic communication throughout the solid porous portions. These pores allow ambient air to be in communication with the interior space of the present invention and thusly ambient air is in communication with a mouthpiece and by virtue of the mouthpiece, the lungs of a user. Unlike apertures, the porous portions create numerous (e.g., thousands of) indirect paths for air to travel through the material. By controlling the porosity and wall thickness of portions of the present invention a smaller, lighter, easier to clean, ergonomically shaped, and overall more simplified breathing resistance device can be created for use.

If the device body is non-porous or has a portion of porous material, then in such embodiments one or more additional porous cassettes can be connected to the device body to create an additional porous barrier between ambient air and a user. If all or a substantial portion of the device body is porous, the cassettes can also still be used and connected to the device body.

With respect to a porous portion, the porosity and wall thickness affect the resistance experienced by a user. Each is hereafter described.

Porosity

As described herein, any portions of the present invention (e.g., the device body or cassette) can have varying porosity. The porosity of a portion of present invention, as defined by a standard porossimeter test, can be between 5 and 200 microns. In certain embodiments, the porosity can be between 5 and 100 microns. In certain embodiments, the porosity can be between 5 and 80 microns. In certain embodiments, the porosity can be between 5 and 50 microns. In certain embodiments, the porosity can be between 5 and 25 microns. In certain embodiments, the porosity can be between 10 and 50 microns. In certain embodiments, the porosity can be between 25 and 100 microns. In certain embodiments, the porosity can be between 25 and 200 microns. In certain embodiments, the porosity can be between 50 and 150 microns. In certain embodiments, the porosity is between 25 and 250 microns

In certain embodiments, the porosity of a porous portion, if any, of a device body or cassette or valve (e.g., a pressure threshold member) or dead space introducing member is selected from: about 5 microns, about 10 microns, about 20 microns, about 30 microns, about 40 microns, about 50 microns, about 60 microns, about 70 microns, about 80 microns, about 90 microns, about 100 microns, about 110 microns, about 120 microns, about 130 microns, about 140 microns, about 150 microns, about 160 microns about 170 microns, about 180 microns, about 190 microns, about 200 microns, about 210 microns, about 220 microns, about 230 microns, about 240 microns, about 250 microns, about 260 microns, about 270 microns, about 280 microns, about 290 microns, about 300 microns.

In certain embodiments, the porosity is less than 300 microns.

In certain embodiments, the porosity is less than 200 microns.

In certain embodiments, the porosity is less than 100 microns.

In certain embodiments the porosity is less than 75 microns.

In certain embodiments, the porosity is between 5 microns and 75 microns.

In certain embodiments, the porosity is between 5 microns and 250 microns.

Material Thickness

In certain embodiments, the thickness of the material that defines a portion of the device (e.g., the device body or cassette) can be between about 0.1 inches and about 2.0 inches. In porous embodiments, the thickness of the porous material is between about 0.1 inches and about 1.0 inches. In certain embodiments, the wall thickness is between about 0.05 and about 0.15 inches. In certain embodiments, the thickness of the porous material is about 0.1 inches. In certain embodiments, the thickness of the porous material is about 0.2 inches. In certain embodiments, the thickness of the porous material is about 0.3 inches. In certain embodiments, the thickness of the porous material is about 0.4 inches. In certain embodiments, the thickness of the porous material is about 0.5 inches. In certain embodiments, the thickness of the porous material is about 0.6 inches. In certain embodiments, the thickness is between 0.1 and 0.6 inches. In certain embodiments, the thickness is between about 0.1 and about 0.15 inches. In certain embodiments, the thickness is between about 0.05 and 0.2 inches. In certain embodiments, the thickness is between about 0.05 and 0.5 inches. In certain embodiments, the thickness is between about 0.05 and 1 inch.

The porous material used in the present invention restricts airflow to provide inhalation resistance, exhalation resistance, or both.

In certain light weight and/or easy to clean embodiments, all or a portion of the present invention can be made from porous plastic. In certain embodiments, polyethylene (PE) ultra-high molecular weight polyethylene (UHMWPE), high-density polyethylene (HDPE), polypropylene (PP), polytetrafluoroethylene (PTFE), and polyvinylidene fluoride (PVDF) are suitable. Ethylene vinyl acetate (EVA), polyethersulfone (PES), polyurethane (PU) and PE/PP co-polymer are also suitable. The material, or a portion thereof, can hydrophobic or hydrophilic. Porous plastic material can be readily obtained from Genpore, 136 Morgantown Rd., Reading, Pa. 19607.

In certain embodiments, the device body (e.g., the frame that is free of any cassettes, dead space introducing members, mouthpiece, etc) of the present invention is non-porous and generally T or L shaped and weighs less than 5 ounces. In certain embodiments, the device body weighs less than 4.5 ounces. In certain embodiments, the device body weighs less than 4.0 ounces. In certain embodiments, the device body weighs less than 3.5 ounces. In certain embodiments, the device body weighs less than 3.0 ounces. In certain embodiments, the device body weighs between 1.0 and 4.0 ounces. In certain embodiments, the device body weighs between 1.0 and 3.0 ounces.

In certain embodiments, when in use the center of gravity of the device (as determined when a device body is free of any cassettes or other components, or as determined inclusive of one or more of a valve, cassette, mouthpiece, dead space introducing member, etc), is located proximal to the lips of a user. In certain embodiments, the center of gravity is less than 1 cm. from the external surface of a user's lips. In certain embodiments the center of gravity is less than 1.5 cm away from a user's lip. In certain embodiments, the center of gravity is less than 2 cm away from a user's lips. In certain embodiments the center of gravity is less than 3 cm away from a user's lips. In certain configurations, the center of gravity is located in front of a user's lips. It is contemplated that in certain configurations the center of gravity of the device of the present invention can be located such that it is inside a user's mouth, proximal to the teeth (e.g., inside the oral cavity). In certain embodiments, it is contemplated that the center of gravity of the device of the present invention is proximal to, or in relative contact with, the lips or mouth of a user.

Inhalation and Exhalation Effort

In certain embodiments, the porosity and wall thickness of the present invention can be varied to provide the following loads experienced by a user during inhalation, exhalation or both. The inhalation effort, exhalation effort, or both, applies to use of the device body alone as well as use of a device body combined with one or more cassettes and/or pressure threshold members (e.g., a fixed or adjustable one way valve).

Respiratory loads can be classified as light, medium and heavy resistance and apply to both flow resistance loading and pressure threshold loading. To the extent there is overlap in ranges, such overlap in characterization may relate to the perceptions of the individual user.

In certain embodiments, the resistance is between 26 and 300 cm. water at a rate (inhalation or exhalation) of 1 liter per second. In certain embodiments the resistance is greater than or equal to 26 cm. water at 1 liter per second. However, in certain embodiments, inhalation resistance can be less, and in some embodiments, inhalation or exhalation resistance is from 10 to 25 cm. water at 1 liter per second. In certain embodiments, the resistance is between 1 and 10 cm. water at a rate (inhalation or exhalation) of 1 liter per second.

Light resistance can be between 26 and 110 cm. water at 1 liter per second. In certain embodiments the resistance is about 35 cm. water at 1 liter per second. In certain embodiments the resistance is about 45 cm. water at 1 liter per second. In certain embodiments the resistance is about 56 cm. water at 1 liter per second. In certain embodiments the resistance is about 65 cm. water at 1 liter per second. In certain embodiments the resistance is about 75 cm. water at 1 liter per second. In certain embodiments the resistance is about 90 cm. water at 1 liter per second.

Medium resistance can be between 26 and 190 cm. water at 1 liter per second. In certain embodiments, resistance can be between 56 and 180 cm. water at 1 liter per second. In certain embodiments the resistance is greater than or equal to about 56 cm. water at 1 liter per second. In certain embodiments the resistance is about 70 cm. water at 1 liter per second. In certain embodiments the resistance is about 100 cm. water at 1 liter per second. In certain embodiments the resistance is about 140 cm. water at 1 liter per second. In certain embodiments the resistance is about 160 cm. water at 1 liter per second. In certain embodiments the resistance is about 170 cm. water at 1 liter per second. In certain embodiments the resistance is about 180 cm. water at 1 liter per second.

Heavy resistance can be between 30 and 300 cm. water at 1 liter per second. In certain embodiments, heavy resistance can be between 150 and 270 cm. water at 1 liter per second. In certain embodiments the resistance is about 150 cm. water at 1 liter per second. In certain embodiments the resistance is about 200 cm. water at 1 liter per second. In certain embodiments the resistance is about 230 cm. water at 1 liter per second. In certain embodiments the resistance is about 250 cm. water at 1 liter per second. In certain embodiments the resistance is about 270 cm. water at 1 liter per second.

In some embodiments, the resistance is greater than 56 cm. water at 1 liter per second. In some embodiments, the resistance is between 56 cm. water and 300 cm water at 1 liter per second.

In some embodiments, one or more of the device body, cassette or valve can impart a vibration or oscillatory effect or flutter on inhalation, exhalation or both. In some embodiments, the vibration is between 6 to 12 Hz. In some embodiments, the vibration is between 20 and 40 Hz. In other embodiments the vibration is greater than 20 Hz. In some embodiments, the vibration is about 5, 6, 7, 9, 10, 12, 13, 14, 15, 16, 17, 18, 19, 20, 22, 24, 25, 30, 35 Hz.

Each of the device body, cassette, valve and dead space introducing member for isocapnic hyperpnea is hereafter described.

Device Body

The device body of the overall device of the present invention typically defines an interior (e.g., hollow) space through which exhaled and inhaled air can pass from external ambient air to a user, typically through a mouthpiece.

In certain embodiments, the body of the device that defines an interior space can be made from any material, such as plastic or metal. In some embodiments, the entirety of the body can be solid, non porous material (e.g., plastic).

However, in other embodiments, at least a portion of the device body is made from porous material, such as ceramic or porous plastic. Such porous embodiments are further described in PCT U.S. Ser. No. 13/54494, the contents of which are incorporated by reference.

In certain embodiments, the present invention includes a rigid or semi-rigid frame, where the frame defines an interior space or hollow portion, and a mouthpiece in gaseous communication with the interior space, where gas or fluid external to the frame can enter the interior space frame during an exhalation or inhalation event of a user. In some embodiments, the frame is generally a T or L shape. The frame can include arms which connect the outer ends of the frame to the center portion of the frame. The center portion of the frame and ends can be configured to fit (e.g., compression fit and/or snap fit) between them one or more modules described herein, such as a valve member, flow resistance loading member, pressure threshold loading member or isocapnic hyperpnea providing member (e.g., a dead space providing member) or measurement member. The outer portion of the end of the frame can also be configured to accept additional modules that extend beyond the ends of the frame.

The frame can be non-porous or optionally include a porous portion.

In certain embodiments, the frame has a Young's modulus between 0.01 and 14 GPa. In certain embodiments, the module is less than 5 GPa. In other embodiments, the modulus is between 0.1 and 4 GPa. In embodiments of a frame having arms and ends, the modulus of the frame should be sufficient to permit flex of the frame to remove attached modules without deforming or damaging the frame.

In certain embodiments, during an inhalation or exhalation event, some or a portion of inhaled or exhaled air can communicate with a mouthpiece without first opening or passing through a one way valve.

In certain embodiments, an arm of the frame can include openings. The openings can be aesthetic in nature or can be functional by permitting visualization of an underlying module. Moreover, the openings can be functional because increasing or decreasing the size of the openings increases or decreases the exposure of the underlying module, thereby potentially altering the functional parameters of the underlying module. In some embodiments, an arm can at least partially envelop a module connected to the frame. In certain embodiments, an arm does not fully envelop a porous module to thereby permit fluid to exit the module under the force of gravity.

Modularity

In certain embodiments, and as described further below, the present invention can be modular. As described above, in certain embodiments, the present invention includes a device body or frame that is adapted to removably receive a breathing related component described herein below, e.g., a porous cassette, a valve (non-adjustable or adjustable) or a dead space introducing member or measuring member. In certain embodiments, the present invention includes a device body, e.g., a frame, that is adapted to removably receive at least two breathing components selected from: porous cassette, a valve (non-adjustable or adjustable), a dead space introducing member or a measurement member. In certain embodiments where two or more breathing components are used in the present invention, the breathing components can be of the same type (e.g., two cassettes) or different types (e.g., a valve and a cassette). Any of the modules can be constructed with features to facilitate the modularity(e.g., inclusion of threads or beads around portions of the modules) such that the modules can interface or mate with complementary structures on the frame.

Modules for Use With The Present Invention

One or more of the following modules may be used, alone, in combination with other modules or in combination with a device body, e.g., a T or L shaped frame embodiment, of the present invention. In some embodiments, a module is a rigid (e.g., substantially non-deformable) body, and/or non-fibrous.

Porous Cassettes

The porous cassettes of whatever type (hereafter “cassettes”) can alter, increase or reduce the inhalation or exhalation effort of a user of a device of the present invention while using a device of the present invention. In certain embodiments, the cassette is a porous plastic cassette.

In certain embodiments, one, two or three or more cassettes having any regular or irregular shape can be used with the device body. In certain embodiments, a cassette can be generally cylindrical or tubular. In certain embodiments, a cassette can further define an interior (e.g., hollow) space such that the interior space of a cassette is in direct communication with an interior space of a frame.

A cassette (or other component for use in the present invention) can be placed in fluid (where fluid is understood to encompass gases or liquids) communication with the interior space of a frame by removable or permanent means such as threaded couplings, snap fittings, adhesives, heat application and the like. Such attachment methods may also be integral, or of one piece construction, with the material or may be adhered to the material though the use of adhesive or sonic welding and the like.

The porosity, wall thickness and overall size can (e.g., 0.5 to 3 inches long, or less than 5 inches and/or greater than 0.5 inches, about 1.5 inches long) can control the resistance to inhalation or exhalation experienced by a user. Thus, inhalation effort and/or exhalation effort of a device of the present invention can be adjusted by varying the porosity and/or wall thickness of cassettes that are in communication with a device body and/or in communication with an interior space of a device body.

Any of the Resistances Described above Can Apply to a Cassette

In certain embodiments, a cassette can be used to create a modular device. In certain such embodiments, one or more cassettes can be stacked, coupled together or arranged in series (e.g., linearly), or in certain designs of a device body (e.g., a T-shaped design) one or more cassettes can alternatively be arranged in a parallel fashion relative to the device body. In other embodiments, one or more cassettes can simultaneously be arranged in both series and also in parallel.

Relative to a user, during inhalation a first porous cassette may be located in series (e.g., upstream) with a second porous cassette having the same or different inhalation resistance. Alternatively, a one way valve cassette (e.g., an adjustable one way valve) may be placed upstream or downstream (e.g., proximally or distally) relative to another cassette of any type.

In certain embodiments, a cassette for use in the present invention has a weight between 0.1 and 1 ounce. In certain embodiments, a cassette weighs less than 0.9 ounces. In certain embodiments, a cassette weighs less than 0.8 ounces. In certain embodiments, a cassette weighs less than 0.7 ounces. In certain embodiments, a cassette weighs less than 0.6 ounces. In certain embodiments, a cassette weighs less than 0.5 ounces. In certain embodiments, a cassette weighs less than 0.4 ounces. In certain embodiments, a cassette weighs less than 0.3 ounces. In certain embodiments, a cassette weighs less than 0.2 ounces.

In certain embodiments of the present invention, a cassette can have an opening on the distal end that can be optionally opened or closed or covered by a user (e.g., by using a snap fit button). The opening can be adapted to receive additional components (e.g., a one way valve) to be placed in series with the cassette (e.g., by virtue of a threaded or snap fit opening). In certain embodiments, a porous cassette can have any shape. In other embodiments, it can have an open “can like” shape, having cylindrical walls having a thickness and a closed end also having a thickness.

The resistance to exhalation can also be increased or decreased by altering the exposure of the total amount of pores to ambient air, porosity, wall thickness, etc. In some embodiments, the arms of an L or T shaped frame can be constructed to increase or decrease the exposure of the porous material, thereby further altering the resistance perceived by a user. In some embodiments, an arm of a frame can cover more than 1% but less than 2%, 5%, 10%, 15%, 20%, 25%, 30%, 35%, 40% or 45% of the cassette.

In certain embodiments, an adjustable cap or other covering can be used to reduce or increase the amount of porous material that is exposed.

Alternatively, in some embodiments a device of the present invention can include one or more apertures in the device body or cassettes which can be covered or uncovered to increase or decrease resistance, respectively.

In certain embodiments, a porous member can impart two resistances depending upon its orientation. In a first orientation, a porous member can provide a first resistance. In a second orientation the porous member can provide a second resistance. Exemplary resistances, in cm H2O, are provided in Table 1 for various porous modules in a first configuration at three different exemplary flow rates and in a second configuration at the same three exemplary flow rates.

TABLE 1

| | Normal Configuration (cmH2O) | | | Reverse Configuration (cmH2O) | | |
|---|---|---|---|---|---|---|
| Module | Avg at 0.5 l/s | Avg at 1.0 l/s | Avg at 2.0 l/s | Avg at 0.5 l/s | Avg at 1.0 l/s | Avg at 2.0 l/s |
| A | 1.07 | 1.41 | 1.94 | 4.61 | 7.32 | 12.57 |
| B | 0.9 | 1.24 | 1.87 | 4.88 | 8.47 | 15.16 |
| 1 | 6.42 | 7.78 | 10.36 | 17.62 | 25.35 | 46.72 |
| 2 | 2.44 | 3.07 | 4.32 | 8.76 | 12.38 | 21.87 |
| 3 | 2.33 | 3.34 | 4.38 | 11.49 | 16.21 | 26.51 |
| 4 | 2.6 | 3.22 | 4.43 | 8.73 | 12.39 | 21.4 |
| 5 | 3.66 | 4.63 | 6.4 | 21.95 | 36.71 | 53.99 |

Adjustable and Non-Adjustable Valve

In certain embodiments, one, two or three or more one way valves can be used to affect the functionality of a device of the present invention by altering, facilitating or easing inhalation effort, exhalation effort or both. In another embodiment, a valve, such as a one way valve (e.g., adjustable or non-adjustable reed or flap type one way valve), a hole or aperture can be included to affect the functionality of the device such that a user can effectively turn off or significantly reduce (by at least 50 to 100%) or substantially eliminate the inhalation or exhalation resistance caused by the device. A one way valve can be integral with the device body or may be connected using connection types apparent to one of skill in the art upon reading this disclosure (e.g., a threaded fit, bayonet fit or adhesive fit). In one embodiment, a one way valve (e.g., an adjustable one way valve) can be removably coupled to a device body and/or substituted for, or used in series or in parallel with, a porous material cassette of the present invention. The one way valve can be included in a body having any shape. In some embodiments, the shape can be a cylinder that is open at both ends or a “can like” structure similar to a porous cassette.

One way valves can be used to select whether inhalation or exhalation of a user undergoes any appreciable resistance, depending upon the orientation of the valve relative to flow of fluid (e.g., air) through the valve. For example, when oriented in a first position, the valve permits inhalation but prevents or substantially impedes exhalation flow through the valve. In a reverse of the first position, when oriented in a second position, the valve permits exhalation but prevents or substantially impedes inhalation flow through the valve. One way valves can also be used to impart threshold resistance loading, in that a threshold load must be exceeded before the valve will open, and the threshold must be maintained in order for the valve to remain open. One way valves can be used alone or in combination with a porous material.

For example a one way valve (adjustable or non-adjustable) can be used and oriented to allow for the rapid release of exhaled air from a user through a valve in the device body and into ambient air. However, upon inhalation the one way valve closes, thus forcing inhalation effort to increase as the inhaled air passes through a cassette, porous portion or other component of the device. In some embodiments, cracking pressures for valves, including non-adjustable valves can be less than: 10 psi, less than 1.0 psi, 0.3 psi, 0.2 psi, 0.15 psi, or 0.09 psi. In certain embodiments, the cracking pressure is less than 0.17 psi. In certain embodiments, the cracking pressure can be the initial cracking pressure necessary for an adjustable one way valve.

In some embodiments, the one way valve can be adjusted so that the valve itself, in addition to a porous portion of the device (e.g., the device body or cassette), provides for variability in resistance by virtue of compression springs, membranes, and the like. Such embodiments provide a resistance (i.e., a respiratory load) and any of the resistances described above with respect to the device body can be used with a valve of the present invention. In certain embodiments, the device body has a resistance to flow that is greater than the resistance to flow of the valve.

Examples of one way valves, adjustable one way valves, or membranes and the arrangement thereof can be found at U.S. Pat. No. 6,726,598 and U.S. Pat. No. 4,739,987, the entire contents of which are hereby incorporated by reference.

In certain embodiments, the housing of a one way valve can be constructed from a porous material as described herein. In other embodiments, the housing of a one way valve is non-porous.

In certain embodiments, a valve for use in the present invention has a weight between 0.1 and 1 ounce. In certain embodiments, a valve weighs less than 0.9 ounces. In certain embodiments, a valve weighs less than 0.8 ounces. In certain embodiments, a valve weighs less than 0.7 ounces. In certain embodiments, a valve weighs less than 0.6 ounces. In certain embodiments, a valve weighs less than 0.5 ounces. In certain embodiments, a valve weighs less than 0.4 ounces. In certain embodiments, a valve weighs less than 0.3 ounces. In certain embodiments, a valve weighs less than 0.2 ounces.

In certain embodiments, a valve can have an opening on the distal end that can be optionally opened or closed by a user (e.g., by using a threaded plug or the like) The opening can be adapted to receive additional components (e.g., a cassette) to be placed in series with the valve (e.g., by virtue of a threaded or unplug-able opening).

Dead Space Introducing Member for Isocapnic Hyperpnea

The average total lung capacity of an adult human male is about 6 liters and females it is about 4.2 liters. Functional residual capacity is the amount of air left in the lungs after normal exhalation. Men leave about 2400 ml on average while women retain around 1800 ml. Thus, men typically exhale 3.6 liters and women typically exhale 2.4 liters. Residual volume is the amount of air left in the lungs after a forced exhalation. The average residual volume in men is 1200 ml and for women is 1100 ml. Vital capacity is the maximum amount of air that can be exhaled after a maximum inhalation, where men tend to average 4800 ml and women 3100 ml.

In certain embodiments, the present invention the device body, alone or in combination with one or more of a cassette or adjustable or non-adjustable valve member, provides or is sufficient to provide a dead space volume. In certain embodiments, in the absence of a dead space introducing member, the volume of the device, alone or in combination with one or more of a cassette or valve member, is insufficient to provide substantial retention/accumulation of carbon dioxide.

In certain other embodiments, in the absence of a dead space introducing member, the volume of the device, alone or in combination with one or more of a cassette or valve member, is sufficient to provide substantial retention/accumulation of carbon dioxide. Thus, in such embodiments, the device body and/or cassette and/or adjustable or non-adjustable valve member function as a dead space forming member.

In other embodiments, the present invention includes a member that introduces a dead space volume, where the dead space introducing member can, in some embodiments, be removably coupled to the device body (e.g. it is a modular component), or the member can be permanently affixed to the device body and/or arranged in parallel or in series with one or more of a cassette or valve as disclosed herein. The dead space introducing member can be any regular or irregular shape, and in certain embodiments it can be tubular. In certain embodiments, the dead space retaining member can be an expandable or elastomeric bag.

The dead space introducing member can be of any material having any parameters as described herein, (e.g., porous or non-porous). The purpose of the dead space volume and/or dead space introducing member is to retain a certain percentage of exhaled gas in the overall internal volume of the device, thereby maintaining an elevated level of carbon dioxide in the device of the present invention with each subsequent inhalation and/or exhalation. Such retention of carbon dioxide can reduce the incidence of hyperventilation. In certain embodiments, the dead space introducing member can be any shape. In other embodiments, the dead space introducing member can be cylindrical or tubular in shape.

In certain embodiments, excluding the amount of exhaled gas that may be present in the porous walls of the device, alone or in combination with one or more of a cassette or adjustable valve member, or dead space introducing member if a porous material is used in the manufacture of the dead space introducing member, the overall dead space is about 2%, 5%, 10%, 15%, 20%, 25%, 30%, 35%, 40%, 45%, 50%, 55% of a typical exhalation volume of a user. In another embodiment, a dead space introducing member alone is sufficient to retain (or increases the overall volume of the total device) about 2%, 5%, 10%, 15%, 20%, 25%, 30%, 35%, 40%, 45%, 50%, 55% of a typical exhalation volume of a user. Thus, in certain embodiments, the dead space introducing member is about 1.08 liters (i.e., 3.6 liters×30%) in volume.

Additional information concerning CO2 measurement can be found in U.S. Pat. No. 4,601,465 and WO2007/089465, the contents of which are incorporated herein by reference in their entirety.

Measurement Member

In some embodiments, the present invention includes a measurement member. A measurement member can be mechanical or electrical and may include or be electrically connected to a memory, a battery (e.g., a rechargeable battery), an optional general purpose processor for performing calculations to determine fitness indicia and to control communication with other devices such as a smartphone or computer by virtue of an optional wireless communicator/transmitter/receiver. In some embodiments, using e.g., the Faraday effect, a small magnet moves against a coil in response to the act of inhaling or exhaling thereby generating a charge that is captured in a capacitor. A logic circuit takes the charge to a rechargeable (e.g., lithium-ion) battery or directly powers a device that is electrical communication with the capacitor.

In one embodiment, a measurement member is or can be connected to another electronic device using a wire or by using standard wireless technology for exchanging data over short distances (e.g., ANT+ and/or using short-wavelength radio waves in the ISM band from 2.4 to 2.485 GHz, or Bluetooth). In one embodiment, the measurement module includes an activator (e.g., a button or switch) to activate the measurement member such that data is stored in a memory or database and/or to establish a wireless connection to another device. In some embodiments, the device can be activated by use upon the sensing of one, two or three or more breaths on or through the measurement member.

In other embodiments, a measurement module optionally includes contacts to permit recharging of the battery and/or communication with another device. In other embodiments, the measurement module includes an inductive charger.

The measurement member can be configured to measure and/or calculate one or more fitness indicia include one or more of pressure, change in pressure, breath count, breath duration, and breath force, maximal inspiratory or expiratory pressure, peak inspiratory or expiratory flow, training load, average power, average inhaled volume, breathing cadence and cumulative breadths. Additional information which can be measured by a measurement member or which can be utilized in accordance with the present invention include time of day, elapsed time, calories burned, CO2 partial pressure, instant pace, average pace over a distance, lap pace over a distance, GPS location/position, elevation change, and elevation. Other indicia are shown in U.S. Pat. No. 8,620,413, and RE44650, the entire contents of which are incorporated by reference, including heart rate. Obtaining heart rate data can be performed by any method, including the use of smart phones, smart watches, chest straps, tablets and the like.

The information (e.g., data) from the measurement member can be stored in a memory (e.g. input into a electronic database) which may be local or distal from the measurement member. The information can be analyzed by an optional processor of the measurement member, or the analysis can be conducted in a device that is in communication with the measurement member. The analyzed information can be optionally stored in a memory and transmitted to another device immediately or at a later time such that the information can be analyzed further and/or displayed to a user and/or healthcare provider or trainer.

Analysis of the information can be used to determine functional data, including one or more of breath count, average number of breaths, average number of breaths over a given time or distance, breath duration, average breath duration, average breath duration over a given time or distance, breath force, average breath force, and average breath force over a given time or distance, temperature, time, CO2 partial pressure and changes thereof, and any of the preceding as a function of one or more of position, pacing, time of day, elapsed time, heart rate, calories burned, CO2 partial pressure, instant pace, average pace over a distance, lap pace over a distance, GPS location/position, elevation change, and elevation. In some embodiments, the notation of a change in pressure in combination with time measurements is sufficient to denote a breath and therefore a breathing rate per unit time. Other indicia which may be used in relation to or analyzed with information obtained from the present invention are shown in U.S. Pat. No. 8,620,413, and RE44650, (e.g., heart rate) the entire contents of which are incorporated by reference. For example, in some embodiments, the data or functional data can be expressed or provided as a function or ratio of a person's (e.g., a user's) heart rate. In other embodiments, functional data may be expressed as a ratio of one or more functional data type to another functional data type and inverses thereof (e.g., elapsed time:heart rate, breath force:pace, average breath force over time:average heart rate over the same or different time)

In one embodiment, the measurement member is or includes one or more of a spirometer, a pressure transducer, a piezo electric transducer and/or capnograph. Other suitable measurement components are known and can be found in U.S. Pat. Nos. 6,314,822, 6,723,055, 8,034,002, 8,620,413, 8,533,620 and RE44650, and U.S. Patent Publication Nos. 20130231574, 20120095352, the entire contents of which are incorporated by reference the contents of each of which are hereby incorporated by reference in their entirety as though fully set forth herein.

The measurement member may be a separate and distinct module which is connectable to or otherwise placed in fluid communication with the frame, alone, in parallel or in series with any module described herein. In another embodiment, a measurement member may be incorporated into any module described herein. In one embodiment, the measurement member is included with a one way valve module. In yet another embodiment, the measurement module is included in the frame (or main body).

In one embodiment, the present invention includes two one way valves modules in fluid communication with the main body or frame and arranged in parallel. A first one way valve provides very low resistance (e.g., less than 1 psi cracking pressure or less than 25 cm of water at 1 liter per minute) upon inhalation and substantially prevents exhalation and a second one way valve that provides very low resistance upon exhalation and substantially prevents inhalation. One or both of the one way valves can include a measurement member. This type of embodiment provides and/or records measurements of fitness indicia (e.g., breath count or breath rate) without creating a substantial resistance to a user and is therefore useful to gauge directly the pulmonary improvement over time obtained from the use of modules that impart resistance during resistance training or improvements obtained from the methods of treatment of disease, as described herein.

Methods of Use and Treatment

The present invention can be used to treat or assist patients with breathing disorders, including patients having COPD, neuromuscular disease, quadriplegia, prevent or treat post operative complications resulting in altered lung function, dyspnea, chronic congestive heart failure, cystic fibrosis, cilliary dysikesia, and other related conditions, as well as slow or reverse the progression of any of the forgoing pulmonary diseases or afflictions. In some embodiments, use of the present invention can prevent pulmonary complications, treat pulmonary complications, improve secretion mobilization, increase FRC, optimize the breathing pattern, and improve oxygenation.

For example, in one embodiment, the method includes identifying a patient at risk of developing a diagnosable disease state or disorder or a patient having a diagnosed disease state and providing a resistance to inhaling or exhaling (e.g., using the devices described herein or any similar functioning device), where the resistance is evaluated for causing the patient to reach a point of stress by observing, but not limited to, symptoms as tiring, shortness of breath and increased pulse rate or other fitness indicia.

The resistance may be then adjusted such that the resistance during a session (e.g., a subsequent session or sessions that occur over a time of hours, day or days, week or weeks or months) is less than or more than that necessary to produce the stress. The resistance can then be reassessed at a later time or session by observing, but not limited to symptoms as tiring, shortness of breath, and increased pulse rate or other fitness indicia on a periodic basis.

Accordingly, one may optionally increase or decrease the resistance, as tolerance to resistance changes; and monitoring the patient for health progression toward or away from a pulmonary disease state, noting that typically the tolerance of resistance diminishes over time/sessions as the patient approaches a diagnosable disease state.

In some embodiments, the initial sessions (e.g., 1 to 5 sessions) can be used to establish a baseline for a patient's pulmonary health and/or rate of progression toward or away from a diagnosable disease state.

It should be noted that a patient at risk of developing a disease state is different than a healthy individual trying to improve performance or a patient that is already diagnosed with a disease state. Specifically, a person is “at risk” when factors (e.g., known injury, preparation for surgery or post-surgery recovery, slow progression of shortness of breath having an as of yet undiagnosed cause, personal or family history or genetic history of a disease state, etc.) indicate that the person or patient is at a greater than normal chance of developing a diagnosable disease state.

Some embodiments can be used alone or in combination with vest therapy, chest physiotherapy, positive expiratory pressure treatments, intrapulmonary percussive ventilation, cough-assist machines and hand-held mechanical percussors.

In some embodiments (e.g., an embodiment making use of a porous member), a direct path to a user's lungs is blocked and accordingly the present invention can be used to filter pollen, dust, pollution and the like during use, thereby limiting or minimizing a user's exposure to such contaminants. After use, the porous member can be easily cleaned (e.g., in a dishwasher) and reused, thereby eliminating the need to continuously purchase surgical masks and the like.

For example, in some embodiments, a module can prevent greater than 80% of particles (e.g., allergens having a particle size of 20-30 microns) from passing through the walls of a module. In other words, in some embodiments, greater than 80% of particles can be prevented from passing from the environment, through a device of the present invention and into a user's body (e.g., lungs).

TABLE 2

| | % Reduction in Pass-Through | | | | |
|---|---|---|---|---|---|
| | of Particles Having a | | | | |
| | Given Size (in Microns) | | | | |
| | 20-30 | 30-40 | 40-60 | 60-80 | >80 |
| Module | 82.8% | 91.8% | 98.9% | 99.99% | 99.99% |
| B, wall | | | | | |
| thickness | | | | | |
| 0.0625 | | | | | |
| inches | | | | | |
| Module | 93.6% | 98.3% | 99.6% | 99.99% | 99.99% |
| B, wall | | | | | |
| thickness | | | | | |
| 0.125 | | | | | |
| inches | | | | | |

Conversely, in certain configurations a device of the present invention can prevent particles that are exhaled by a user from exiting the device and/or entering a local environment. In some embodiments, the % of reduction of pass-through can be greater than: 70%, 75%, 80%, 82%, 85%, 90%, 91%, 92%, 93%, 95%, 98%, 99%, 99.9%, 99.99%.

Particles can be of any size, but are typically accepted to have the following particle size characteristics (in microns):

Pollen: 10-100

Mold Spores: 6-90

Dust Mite Allergens: 0.08-12

Germs: 0.5-10

Cat Allergens: 0.09-5.0

Heavy Dust: 90-1000

Settling Dust: 0.8-100

Atmospheric Dust: 0.001-1

Soot: 0.008-0.4

Viruses: 0.004-0.06

It is understood that by modifying a device of the present invention (e.g., porosity, wall thickness, etc.) different particle sizes can be prevented from passing from the external environment and into a user, and vice versa.

In certain embodiments, use of a device, particularly a filtering aspect of a device of the present invention can increase compliance and/or perseverance in a given treatment protocol because the patient improves and/or does not inhale allergens, etc., which may hinder use of a non-filtering aspect of a device.

The present invention can also be used to assist breath training in an athletic context to increase the strength and efficiency of breathing. In some embodiments, the resistance to inhalation, exhalation, or both, can be set such that a user can use an embodiment of the present invention for between 3 to 300 minutes per day. In some embodiments, a user can use the present invention periodically or continuously throughout an entire day. In some embodiments, a user can use the present invention for 10 to 20 minutes per day. In some embodiments, a user can use the present invention for more than 5 minutes per day. In some embodiments, a user can use the present invention for more than 20 minutes per day.

In some embodiments, the present invention can be used in a manner consistent with resistance training. In some embodiments, such resistance training includes a time period of exhalation and/or inhalation resistance or a predetermined number of breaths through a device of the present invention followed by a period of rest wherein the device of the present invention is not used. In some embodiments, the time period of breathing with resistance is selected from about 10 seconds, about 15 seconds, about 20 seconds, about 25 seconds, about 30 seconds, about 45 seconds, about 60 seconds, 90 seconds, 120 seconds, about 10 seconds to 40 seconds, about 10 seconds to about 60 seconds, about 10 seconds to 90 seconds, about 10 seconds to 120 seconds.

In certain other embodiments, the predetermined number of breaths of a training set include at least 5, at least 10, at least 12, at least 15, at least 20, at least 25 at least 30, at least 40, between 5 and 15, between 10 and 20 breaths, between 20 and 40 breaths.

In other embodiments, the rest period is selected from about 10 seconds, about 15 seconds, about 20 seconds, about 25 seconds, about 30 seconds, about 45 seconds, about 60 seconds, 90 seconds, 120 seconds, about 10 seconds to 40 seconds, about 10 seconds to about 60 seconds, about 10 seconds to 90 seconds, about 10 seconds to 120 seconds, about 6 hours, about 8 hours about 12, and between 8 and 12 hours. When used in a manner consistent with resistance training a user is typically not physically active (e.g., not running, biking, playing a sport or otherwise training, or in the alternative a user is sitting, driving or is generally not engaged in cardiovascular or muscular training during use of the device) and the user is focusing primarily on pulmonary exercise. The user can complete 1, 2, 3, 4, 5, 6, 7, 8, 9, or 10 sets with a rest period between each set. In some embodiments, a user can complete 3 or more sets, with a rest period between each set.

In certain embodiments, and as described below, the present invention is ergonomically formed, small enough and/or light enough that it can be used for or during endurance training or while exercising, e.g., while running, stair climbs, cycling, weightlifting, engaging in aerobics, walking, rock climbing, or swimming with a kickboard or other activity.

Alternatively, the device of the present invention can be used for much longer periods, e.g., for endurance training to impart or provide hill climb or high altitude training intensities. In such embodiments, the device of the present invention may be used while a user is relatively inactive (e.g., resistance training over an extended period) or while the user is actively covering/traversing a certain distance.

In one embodiment, a user may employ the present invention in the course of inhaling, exhaling, or both, for an extended period of time. In one embodiment, the device of the present invention may be used for about 3 minutes, about 4 minutes, about 5 minutes, about 10 minutes, about 15 minutes about 20 minutes. In some embodiments the present invention may be used for between 10 minutes and 60 minutes. In other embodiments a user can use the present invention for a time period greater than an hour.

In some endurance related embodiments, a user may actively traverse a distance while the user is also engaged in cardiovascular exercise, where the distance is selected from: about 10 yards, about 100 yards, at least a quarter mile, at least a half mile, at least a mile, at least 3.1 miles, at least 6.2 miles at least 10 miles, at least 13.1 miles, at least 20 miles, at least 26.2 miles, at least 30 miles, at least 50 miles, from 1 mile to 10 miles, from about 3.1 miles to 13.1 miles from about 1 mile to 20 miles.

In some other embodiments, a user may actively undertake an elevation change while using the device. In such embodiments, a user may ascend at least: about 10 feet, about 20 feet, about 30 feet, about 50 feet, about 100 feet, about 500 feet, about 1000 feet, or about 5000 feet

In certain embodiments, the device of the present invention can be configured to provide flow resistive loading for inhalation and exhalation. In certain embodiments, the device of the present invention can be configured to provide flow resistive loading for inhalation only. In certain embodiments, the device of the present invention can be configured to provide flow resistive loading for exhalation only In certain embodiments, the device of the present invention can be configured to provide pressure threshold loading for inhalation and exhalation. In certain embodiments, the device of the present invention can be configured to provide pressure threshold loading for inhalation only. In certain embodiments, the device of the present invention can be configured to provide pressure threshold loading for exhalation only. In certain embodiments, the device of the present invention can be configured to provide pressure threshold loading for inhalation and flow resistive loading for exhalation. In certain embodiments, the device of the present invention can be configured to provide flow resistive loading for inhalation and pressure threshold resistance for exhalation. Isocpanic hyperpnea (e.g., by using a dead space introducing member if the device of the present invention does not provide sufficient dead space already), can be used in combination with any of the forgoing configurations. A measurement member may be used in combination with any of the forgoing embodiments.

In some embodiments, the present invention may further include a carbon or other air filter insert within the inner diameter or cover over the outside or at one or more ends of a module or located in fluid communication with a user (e.g., in the frame). In certain embodiments, a module can be replaced with a carbon or other air filter with the same dimension. In use, the addition of the filter to an embodiment of the invention is that a user can still use the device, with or without resistance, in the presence of air pollutants. As such, certain embodiments of the invention have filtration aspects and may not require additional carbon or other air filter inserts.

In some embodiments, the present invention includes a device of the present invention and instructions for its use in accordance with any of the methods described herein, where the device and instructions are arranged in a kit.

Outcome Based Methods

In certain embodiments, the present invention can include a timer that maintains a count of each session within a specified period of time (e.g., an hour or a day or two days or a week or less or more) for showing the number of training/treatment sessions/intervals and other descriptive statistics. In certain embodiments, as detailed above, the present invention can include a wireless communication member to communicate results to a receiving device (e.g., a handheld computer or physician's computer or server).

In some embodiments, a device of the present invention can communicate, via wire or wirelessly, with other computing devices (e.g., phones, tablets, desktops, servers, etc.,) for recording and/or use. Similarly, a device or computing device in communication with the present invention can selectively or automatically transmit data (as detailed herein), to a physician or EMR/EHR system or personal trainer directly or indirectly through, e.g., a health information exchange such that the physician or trainer can comment on or provide adaptations to the training regimen.

In certain embodiments, the present invention can be used in combination with electronics which provide incentives to a user/patient to improve his/her breathing and/or ventilation. In certain embodiments, the present invention can be used in combination with electronics to provide incentives (light, sound, tactile vibration) to prevent the onset of an anticipated respiratory condition. For example, such devices can be used immediately after surgery (e.g, abdominal or cardiovascular surgery) as part of post operative care to prevent the onset of respiratory distress.

In one embodiment, the present invention provides for audio and/or visual feedback (e.g, a transducer) and data recordation (e.g., actual patient/user performance and/or results) based on achieving, or maintaining for a time period, a specified and/or selected therapeutically effective outcome, such as breathing rate vs. pressure level vs. threshold level vs. number of breathing sets, etc. In such embodiments, the user obtains feedback ((light, sound, tactile vibration) relative to a predetermined standard, but the actual performance of the user can be tracked and then compared relative to the standard. In certain embodiments, feedback and/or music or other information can be provided to a user through bone conduction via a mouthpiece of a device of the invention.

In some embodiments, when one or more of data, functional data, or a ratio thereof meets or exceeds a predetermined threshold requirement, an alarm signal can be sent to a user or remote monitor (e.g., a healthcare provider or trainer). For example, when the ratio of breathing rate to heart rate is unexpectedely reduced, such reduction may be indicative of a medical event or emergency and accordingly, an alarm can be sent to the user or a remote monitor to provide assistance to the user.

In certain embodiments, the present invention can include an adaptive intelligence (e.g., software updates) such that controls and/or feedback incorporates one or more of data and/or results and/or methods from studies or recommended medical regimens (e.g., breathing rate vs. pressure level or threshold level vs. number of sets) for treatment of a given indication (e.g., asthma or COPD) and the patient's/user's profile (e.g., age, weight, health status) and/or the patient's/user's current or actual performance on the device. In accordance with such embodiments, the personalized data that is applicable to a particular user or set of users, the data obtained over time can be tracked and the use regimen can be changed or adjusted based on user progression over time in view of a particular condition and/or profile/demographic of a user. The obtained empirical data set, whether in view of a single user or in view of data generated by a plurality of users, can be used or analyzed to thereby customize a use regimen to optimize an outcome (e.g., clinical outcome or athletic goal outcome). Based on the regimen actually used, the present invention can provide real time collection of data of a user for rapid integration and adaptation of treatment or training regimen.

In other words, such systems and/or data can be used to aggregate data across patient data and results to permit a user or a third party (e.g., physician, physical therapist, personal trainer) or determine and/or optimize a use regimen in view of one or more of the patient's/user's profile, indication, a patient's/user clinical outcome. Such embodiments can be useful to facilitate remote monitoring or tele-medicine type interactions between a patient and third party (e.g., care provider or trainer).

For diagnostic purposes, embodiments of the present invention can include sensors for the detection of biomarkers of a disease state, including cancer, asthma, cystic fibrosis and lactose intolerance. In one embodiment, a disease can be detected by virtue of elevated alkanes such as pentane, carbon monoxide and/or nitric oxide—including FeNO where the nitric oxide content of an asthmatic's breath can be as high as 100 or 200 parts per billion, in exhaled breath as well as detecting biomarkers for other disease states (e.g., cystic fibrosis through the detection of nitrite levels in exhaled breath condensates). In such embodiments, the present invention can include materials embedded or attached to the surface of the porous plastic which in turn undergo a change in state (e.g., change in color or a colorimetric sensor array, change in electrical resistance) when contacted with a biomarker. Suitable materials for the detection of biomarkers include ozone, amperometric microelectrode assays (such as those disclosed in U.S. Pat. No. 8,485,983, the entire contents of which are incorporated by reference in its entirety). Other suitable detection materials may be obtained from Metabolomx in Sunnyvale, California. Thus, in one exemplary embodiment, a porous plastic member includes an amperometric microelectrode assay.

Given the substantial surface area of a porous plastic components of the present invention, large amounts of detection material can be included in some embodiments of the present invention to increase accuracy of detection of the disease state biomarkers. However, such materials can be included on other components of the present invention, including a frame, pressure threshold component, dead space providing member, or flow resistive loading member or mouthpiece.

Such diagnostic embodiments permit both ongoing monitoring and diagnosis of a disease state, but also the treatment of the disease state itself through pulmonary exercise. A device of certain embodiments of the present invention will be especially suitable for use by a wide range of compromised individuals, such as the very elderly, young children and otherwise incapacitated patients.

Other Components

In some embodiments, the present invention can include a nose clip and or nose clip holder to close the nose of user and thereby force a user to breathe solely through the user's mouth and therefore solely through the device of the present invention. The nose clip can be separate or integrated with the device of the present invention. Alternatively, the present invention can be used in combination with a nasal inspiratory resistance trainer such as that shown in U.S. Publication No. 20130157810, the contents of which are incorporated by reference.

In other embodiments, the present invention includes two raised nubs which function to plug the nose of a user. The raised nubs can be solid plastic, a soft material or porous plastic, or any other material suitable for at least partially blocking the nasal passageway of a user.

Other embodiments may also include one, two or three or more clip portions or eyelets which permit the attachment of a necklace or string to a portion of a device of the present invention to permit a user to simply spit out the device of the present invention (or in case the device of the present invention falls out of a user's mouth during exercise) such that the present invention can hang around a user's neck, thereby permitting continued hands free operation and reducing the incidence of inadvertent loss of the device. Accordingly, in one embodiment, the present invention includes the method of a user releasing the device of the present invention (e.g., from the user's mouth) while still retaining possession of the device. In certain other embodiments, the necklace or string can include an elastomeric material so as to facilitate retention of the device of the present invention in a user's mouth. Specifically, the elastomeric material (e.g., rubber or elastic) provides tension support around a user's head. However, in certain embodiments, such support permits the easy removal of the device of the present invention from a user's mouth by one or more of spitting, by hand, or merely mouth relaxation (e.g., in the case of loss of consciousness).

In certain porous embodiments, the porous nature of the present invention may also permit additional constituents to be adhered to a porous portion. In such embodiments, detection of disease, e.g., liver disease, gastrointestinal disease, diabetes or cancer, can be accomplished by contacting a surface with appropriate detection agents such that disease biomarkers in saliva and exhaled air (e.g., nitric oxide, acetone, volatile organic compounds) can be detected (e.g., by a change in color) upon contact of the disease biomarker with the detection agents.

In certain other embodiments, the present invention includes a porous member alone or in combination with a flexible sheet (e.g., neoprene), to there by provide resistance. In such an embodiment one or more of the porous portions described herein can be integral with such a flexible sheet, such that the sheet forms a mask to be worn by a user, such that the mask forms at least a partial seal on a user's face to thereby cause the user to breathe through the porous member.

In certain embodiments, the present invention does not provide a seal for the nose, or is free of nose sealing, where should such sealing be necessary, such sealing can be providing by a secondary component, e.g., a nose clip.

In certain embodiments, the components of the device of the present invention (e.g., the device body or cassette) can be constructed free of adhesives. In some embodiments, the portions of the overall device of the present invention may be welded (e.g., sonically welded) together or friction fit together.

In some embodiments, a device of the present invention is cleaned to a pharmaceutically acceptable level.

### Non Limiting Exemplary Embodiments

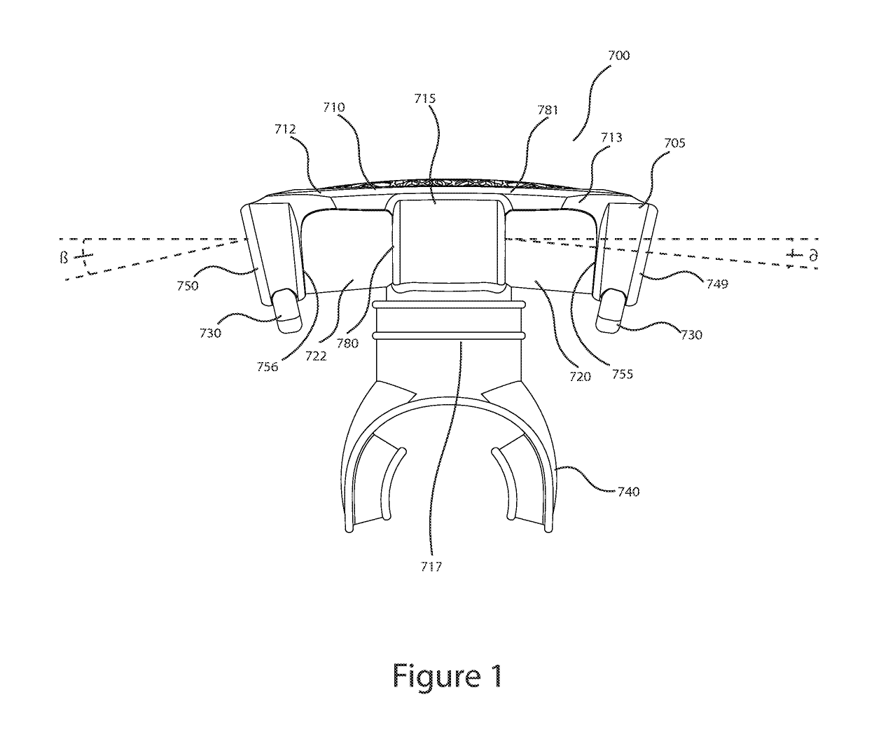

Referring to FIGS. 1 through 7, there is illustrated various non-limiting embodiments of the present invention.

FIG. 1 shows a top down view of a rigid but slightly deformable plastic, frame based device 700 of the present invention. Frame 705 is generally T shaped and is configured to support modules 720 and 722. Left and right arms 712 and 713 connect frame center portion 715 to ends 749 and 750, respectively. Arms 712 and 712 and 713 can also further define front face 710. Frame center 715 includes attachment interfaces 780 and 781, hollow portion interior space 717 in fluid communication with mouthpiece 740 and modules 720 and 722 which connect to the center portion at interfaces 780 and 781. Interfaces 780 and 781 can, in some embodiments, have an angle alpha to impart an angle or provide an illusion of a curve to better match the curvature of a user's face. In some instances angle alpha can be between 1 and 20 degrees. In other embodiments, there is no angle. In certain embodiments, electronics, including one or more of pressure sensors, flow rate sensors, timer, GPS, audio or visual alarms, etc, may be incorporated into frame 705.

Ends 749 and 750 can also include eyelets 730. In the embodiment shown, module 720 is a porous plastic module having a porosity between 5 and 300 microns. Whereas module 722 is a non-porous one way valve module. The ends of modules 720 and 722 are bound by ends 749 and 750, respectively and are held in place by a interference or snap fit at end internal interfaces 755 and 756. The distal portion of ends 749 and 750 can have an angle beta to further impart a curve or provide an illusion of a curve in the event that additional modules are coupled to the frame 705. Angle beta can be between 1 and 20 degrees. In some embodiments there is no angle beta.

Angles alpha and beta are typically measured from a centerline toward a user's face or toward the mouthpiece, as shown. However, the angles can be imparted in any direction, including down, up and forward.

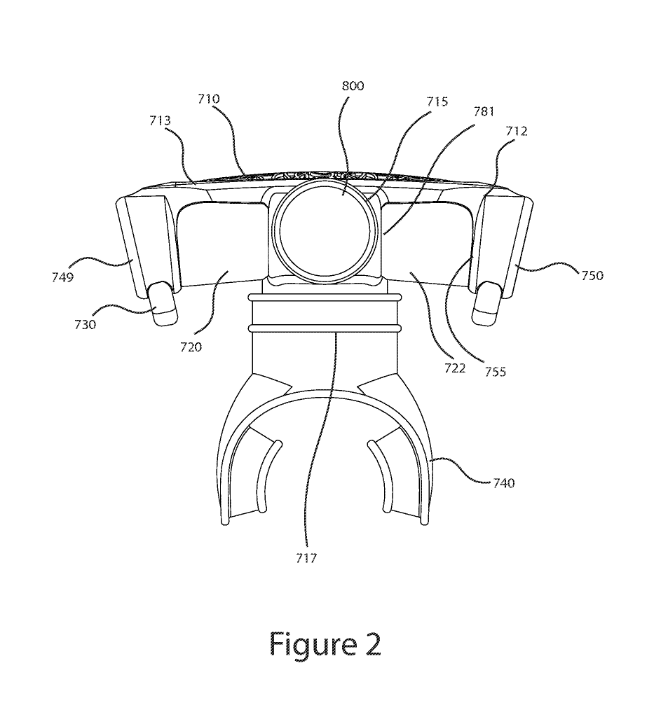

FIG. 2 shows a bottom up view of a device 700 of the present invention. Button 800 is clearly defined. Button 800 can be used to insert into external ends interfaces 751 or 752, as shown in FIG. 10.

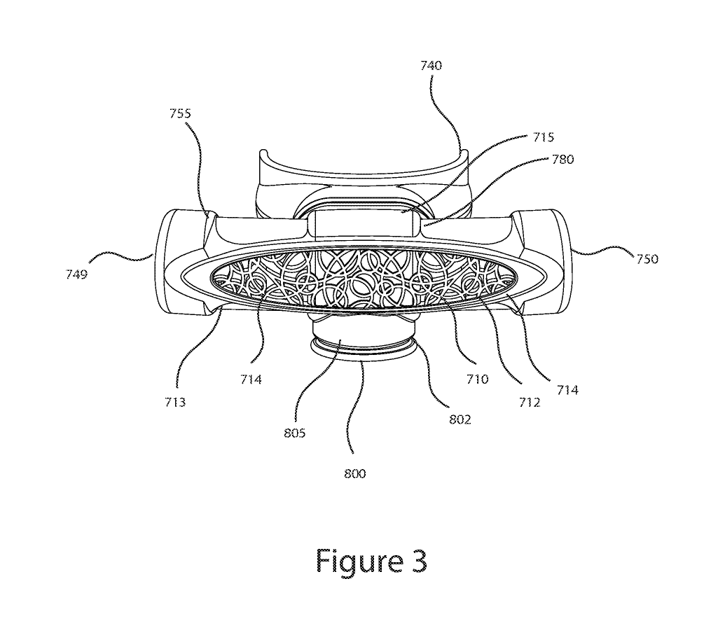

FIG. 3 shows a front view of a device 700 of the present invention. Arms 712 and 713 define front face 710 having openings 714. Also, button interface 802 of button 800 permits removable insertion of button 800 into the complementary structure of external ends interfaces 751 or 752. In certain embodiments, electronics, including one or more of pressure sensors, flow rate sensors, timer, GPS, audio or visual display, tactile/vibration alarms, etc, may be incorporated into button 800. A sensor according to the present invention may also extend into interior space 717. In one embodiment, button 800 includes a sensor (e.g., a pressure sensor available from Honeywell or other sensor type device) that fits into an aperture (not shown) formed on the body of device 700 that permits the sensor to enter into the interior space 717 of device 700. In some embodiments, the aperture can be between 1 and 10 mm in diameter. In other embodiments the aperture can be between 1 and 5 mm, in other embodiments, the aperture can be about 3 mm in diameter.

In certain embodiments, device 700 optionally includes an opening or fitting, not shown, adapted to receive a pharmaceutical delivery system or external device such that upon use of the delivery system, the drug can enter interior space 717 of device 700 to permit administration of the drug to a user. In other embodiments, device 700 includes an opening and/or fitting, not shown, adapted to permit enrichment gas (e.g., oxygen, and/or helium and/or CO2) to enter interior space 717 of device 700 to then be administered to a user before, during or after use of a device of the present invention.

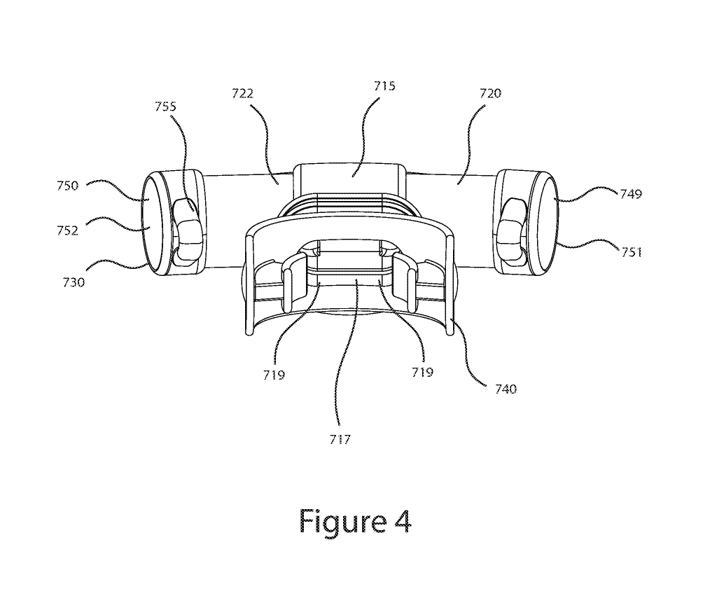

FIG. 4 shows a mouthpiece view of a device 700 of the present invention. External ends interfaces 751 or 752 permit connection of additional modules to place such modules in fluid communication with center 715 and interior space 717. Also shown are optional vanes 719 which direct breath air inside interior space 717.

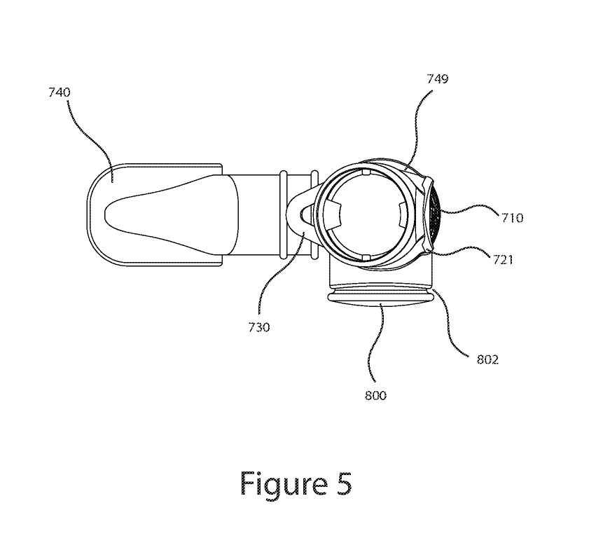

FIG. 5 shows a right side view of a device 700 of the present invention, where porous plastic module 721 has a closed end.

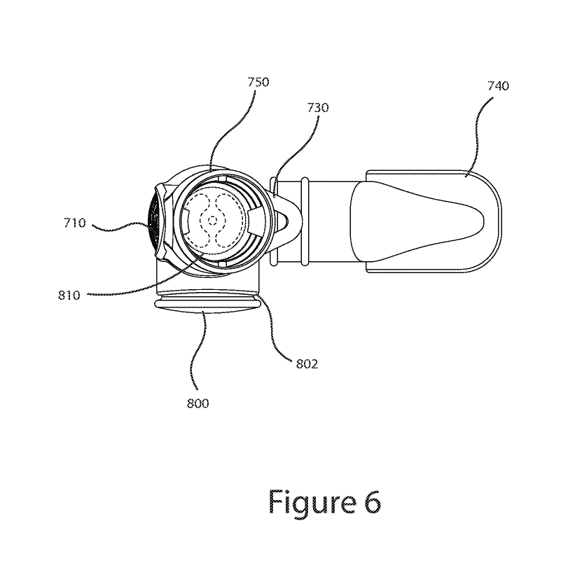

FIG. 6 shows a left side view of a device 700 of the present invention, where one way valve 810 inside of module 722 can be seen. In the shown configuration, the module is arranged to permit exhalation but resist inhalation. Therefore, a user will perceive resistance during inhalation by virtue of porous plastic module 720 but little to no resistance upon exhalation. The modules 720 and 722 can be reversed as to sides and direction. For example, module 722 can be reversed to permit resistance on exhalation, thereby imparting a resistance upon a user during exhalation through module 720.

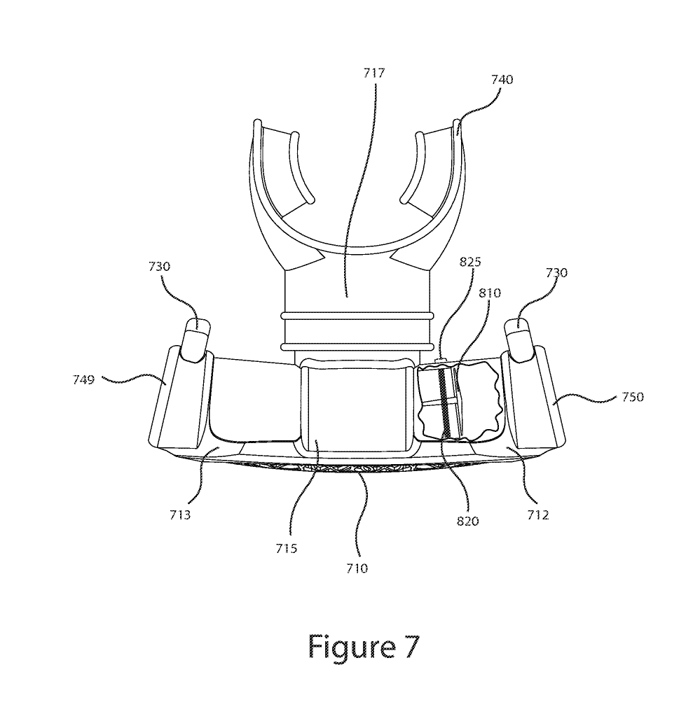

FIG. 7 shows a partial transparent view of module 722. Valve 810 is arranged proximal to piezo electric measurement member 820 having communication and charging point 825. In certain embodiments, electronics, including one or more of pressure sensors, flow rate sensors, timer, GPS, audio or visual alarms, etc, may be incorporated into button module 722. Such electronics can be incorporated into any feature or component of the claimed invention, including resistance modules and isocapnic hyperpnea modules.

As used herein, the term “about” means + or −10% of the value referenced, inclusive of the value referenced. Thus “about 10” is understood to fully support both “10”, as well as the range of “9 to 11” Moreover, the term “about” is understood to be optionally applicable to every value set forth herein, whether or not explicitly stated as such in this disclosure. Thus, for example, a value of “10%” set forth herein is understood to support a claim limitation of “10%” as well as a claim limitation of “about 10%.”

It is also understood that individual values provided as part of a listing of values may be selected individually. For example, “2%, 5%, 10%, 15%, 20%, 25%, 30%, 35%, 40%, 45%, 50%, 55%” is understood to mean that any particular value may be selected from the list and used or claimed alone, e.g., 2% or 30%, 55% or about 55%).

In this disclosure, there is shown and described various embodiments of the invention. However, as aforementioned, the invention is capable of use in various other combinations and environments and is capable of changes or modifications within the scope of the inventive concept as expressed herein. Various embodiments may be altered or combined with other embodiments without changing or altering the scope of this disclosure.

Each and every reference set forth in this disclosure is hereby incorporated by reference in its entirety as if set forth fully herein.

What is claimed is:

1. A pulmonary exercise device comprising frame having a center portion defining an interior space; the frame having a first attachment interface in fluid communication with the interior space, and a second attachment interface connected to the center portion by an arm portion; and optionally a mouthpiece in fluid communication with the interior space.

2. The device of claim 1, wherein the frame comprises a T or L shape.

3. The device of claim 1, further comprising one or more of a valve member, a resistance member or measurement member in fluid communication with the interior space and supported by at least one attachment interface.

4. The device of claim 3, wherein two or more of a valve, a resistance member or measurement member in any combination are in fluid communication with the center portion.

5. The device of claim 3, wherein one or both of the valve and resistance member or measuring member are elongated, tubular, supported by and removably connected to the frame at or more two contact points.

6. The device of claim 3, wherein the resistance member comprises one of a porous plastic module, an aperture providing member, an adjustable valve, or a dead space providing member.

7. The device of claim 3, wherein the measurement member comprises one or more of mechanical or piezo electric measurement member configured to measure one or more of exhalation count, inhalation count, breath count, duration and/or force thereof.

8. A pulmonary exercise device comprising a device body adapted to removably receive:
(a) a one-direction valve having a cracking pressure of less than 10 psi; and, (b) at least one other component selected from a one direction valve having a cracking pressure less than 10 psi, a porous plastic cassette, an adjustable valve, a measurement member and a dead space introducing member.

9. The device of claim 8 wherein one or more of a valve, cassette or dead space introducing member further includes a pressure measurement member.

10. The device of claim 8 wherein the measurement member comprises piezoelectric pressure sensor in communication with a battery, a processor and a memory.

11. Use of the device of claim 10 to generate data, wherein the data comprises one or more of time of day, elapsed time, GPS location, CO2 partial pressure, breath count, breath duration, and breath force.

12. Use of the device of claim 11, wherein the data is manipulated to generate functional data providing one or more of breath count, average number of breaths, average number of breaths over a given time or distance, breath duration, average breath duration, average breath duration over a given time or distance, breath force, average breath force, and average breath force over a given time or distance and wherein the functional data is stored in a electronic database.

13. Use of the device of claim 12, wherein the data or functional data are stored in a memory as a function or ratio of heart rate.

14. A training method for a patient at risk of developing a diagnosable pulmonary disease state comprising observing a patient engage in a pulmonary exercise session by inhaling or exhaling through a resistance or dead space imparting member;
assessing said resistance to inhaling or exhaling required to cause the patient to reach a point of stress by observing symptoms including one or more of tiring, shortness of breath and increased pulse rate.

15. The training method of claim 13, further comprising providing instructions to adjust or adjusting the resistance such that the resistance throughout each session is less than that necessary to produce said stress;
reassessing a resistance required to cause the patient to reach the point of stress by observing symptoms including one or more of tiring, shortness of breath, and increased pulse rate on a periodic basis; and
optionally increasing or decreasing the resistance for a subsequent session as tolerance to resistance changes.

16. The training method of claim 13, wherein the session is at least once daily.

17. The training method of claim 13, wherein the training slows the progression of a pulmonary disease.

18. The training method of claim 17, wherein the disease is COPD.

19. The training method of claim 13, further comprising the step of monitoring the patient for health progression toward or away from a pulmonary disease state.

20. A composition, use or method according to any of the claims 1-19, wherein use of the device inhibits the inhalation of pollen sized particles while also providing a predetermined resistance during inhalation.
